(19) United States
(12) Patent Application Publication
(10) Pub. No.: US 2019/0012930 A1
(43) Pub. Date: Jan. 10, 2019

(54) METHODS FOR FACILITATING ADDICTION RECOVERY SERVICES AND DEVICES THEREOF

(71) Applicant: Brian McAlister, Fairfield, NJ (US)

(72) Inventor: Brian McAlister, Fairfield, NJ (US)

(21) Appl. No.: 16/029,836

(22) Filed: Jul. 9, 2018

Related U.S. Application Data
(60) Provisional application No. 62/530,333, filed on Jul. 10, 2017.

(51) Int. Cl. G09B 19/00; G16H 80/00; G09B 5/06

(52) U.S. Cl. CPC G09B 19/00; G09B 5/065; G16H 80/00

(57) ABSTRACT

Methods, non-transitory computer readable media, and client devices are disclosed that authenticate a user in response to a received login request, output a mood selector tool, and retrieve current mood data based on a received interaction with the mood selector tool. User data is obtained for the user and a dashboard is generated and output. The dashboard comprises selectable menu options. A selection of one of the selectable menu options is received. One of a plurality of modules is output corresponding to the one of the selectable menu options. The one of the modules is configured to provide content based on at least a portion of the user data and the current mood data. A determination is made when a notification condition exists and a push notification is output, or an automated message is sent, to a third party, when the determination indicates that a notification condition exists.

### CROSS-REFERENCE TO RELATED APPLICATION(S)

This application claims the benefit of U.S. Provisional Patent Application Ser. No. 62/530,333, filed on Jul. 10, 2017, which is hereby incorporated by reference in its entirety.

### FIELD

This technology generally relates to methods and devices for improved dynamic delivery of media content to facilitate recovery management services.

### BACKGROUND

Current addiction recovery services and treatment programs have limited effectiveness leading to several negative personal and societal consequences. In particular, treatment programs are often not tethered or tailored to individuals to facilitate effective monitoring, are not responsive or adaptive to current or immediate needs of an individual, and do not deliver content in an adaptive and appropriate manner for particular individuals. Moreover, it is currently difficult to obtain valuable metrics regarding individual progress that can be used to improve a treatment program and associated outcomes.

### SUMMARY

Methods, client devices, and non-transitory computer readable media are disclosed that authenticate a user in response to a received login request, output a mood selector tool, and retrieve current mood data based on a received interaction with the mood selector tool. User data is obtained for the user and a dashboard is generated and output. The dashboard comprises a plurality of selectable menu options. A selection of one of the selectable menu options is received. One of a plurality of modules is output corresponding to the one of the selectable menu options. The one of the modules is configured to provide content based on at least a portion of the user data and the current mood data. A determination is made when a notification condition exists and a push notification is output, or an automated message is sent, to a third party, when the determination indicates that a notification condition exists.

Methods, recovery management server devices, and non-transitory computer readable media are disclosed that monitor utilization of a recovery management application and obtain user and for a plurality of users of the recovery management application over a particular time period based on the monitoring. The user data comprises information submitted via a plurality of modules, intake interview data, or statistical usage data. Progress data is obtained for the users. The progress data is correlated with the user data to identify key parameters based on efficacy. Functionality of the recovery management application is modified based on the key parameters in order to improve outcomes for the users.

This technology has a number of associated advantages including providing methods, devices, and non-transitory computer readable media that facilitate more efficient recovery management services. With this technology, addiction recovery services are more effectively facilitated via integration of a recovery management application with multimedia and other content and resources in order to improve the likelihood that a recovering addict will remain on a path of sobriety.

### BRIEF DESCRIPTION OF THE DRAWINGS

FIG. 1 is a block diagram of an exemplary network environment with a recovery management server device and client devices;

FIG. 2 is a flowchart of an exemplary method for integrating relevant multimedia content and providing notifications and other resources to assist users in maintaining sobriety;

FIG. 3 is a screenshot of an exemplary mood selector tool;

FIG. 4 is a screenshot of an exemplary video module;

FIG. 5 is a screenshot of an exemplary goals module;

FIG. 6 is a screenshot of an exemplary progress module;

FIG. 7 is a flowchart of an exemplary method for automated updating of a recovery management application based on machine learning to improve efficacy and associated user outcomes; and

FIG. 8 is a block diagram of exemplary portals or interfaces provided by an exemplary recovery management server device.

### DETAILED DESCRIPTION

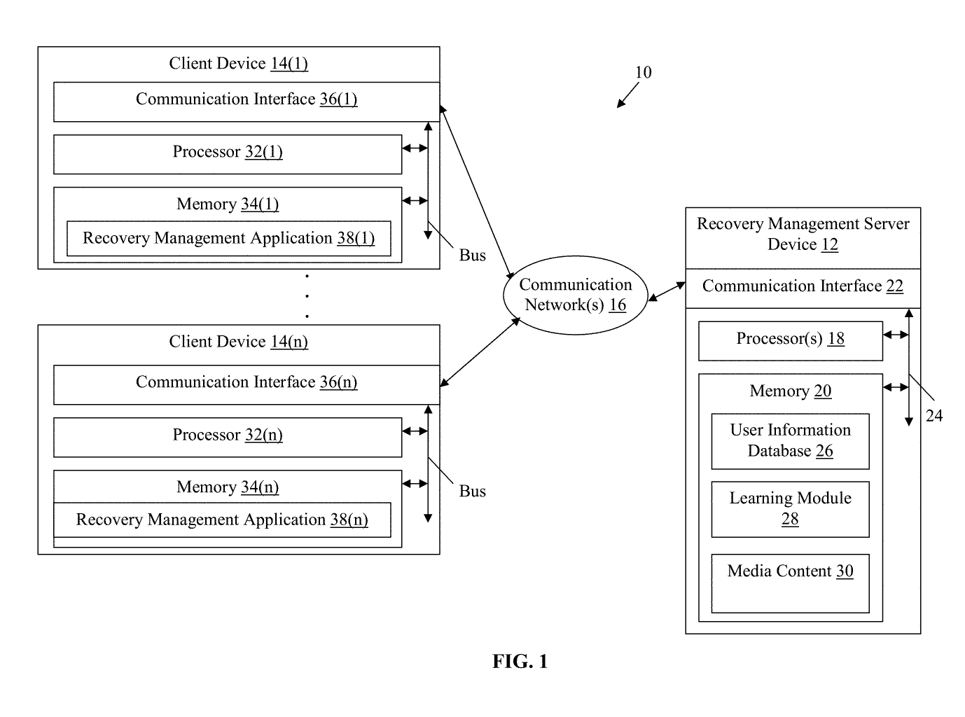

An exemplary network environment 10 with a recovery management server device 12 coupled to client devices 14(1)-14(n) by communication network(s) 16 is illustrated in FIG. 1, although this network environment 10 can include other numbers and types of systems, devices, and elements in other configurations. While not shown, the network environment also may include additional network components such as routers and switches, which are well known to those of ordinary skill in the art and thus will not be described here. This technology provides a number of advantages including methods, non-transitory computer readable media, and devices that more effectively facilitate addiction recovery services via integration of a recovery management application with multimedia and other content and resources in order to improve the likelihood that a recovering addict will remain on a path of sobriety.

The recovery management server device 12 in this example includes processor(s) 18, a memory 20, and a communication interface 22, which are coupled together by a bus 24 or other communication link, although other numbers and types of systems, devices, components, and elements in other configurations and locations can also be used. The processor(s) 18 in the recovery management server device 12 execute a program of stored instructions for one or more aspects of the present technology, as described and illustrated by way of the examples herein, although other types and numbers of processing devices and configurable hardware logic could be used and the processor(s) could execute other numbers and types of programmed instructions.

The memory 20 in the recovery management server device 12 stores these programmed instructions for one or more aspects of the present technology as described and illustrated herein, although some or all of the programmed instructions could be stored and executed elsewhere. A variety of different types of memory storage devices, such as a RAM, ROM, flash, SSDs, HDDs, or other computer readable medium which is read from and written to by a magnetic, optical, or other reading and writing system that is coupled to the processor(s), can be used for the memory 20.

In this example, the memory 20 includes a user information database 26, a learning module 28, and media content 30, although other types of applications, modules, and/or databases could also be included in the memory 20 in other examples. The user information database 26 stores information for a plurality of users of a recovery management application that is provided or hosted by the recovery management server device 12. The information can include information previously submitted via one or more modules of the recovery management application, user profile data, intake interview data, and/or statistical usage data relating to the recovery management application, for example, and other types of information can also be stored in the user information database.

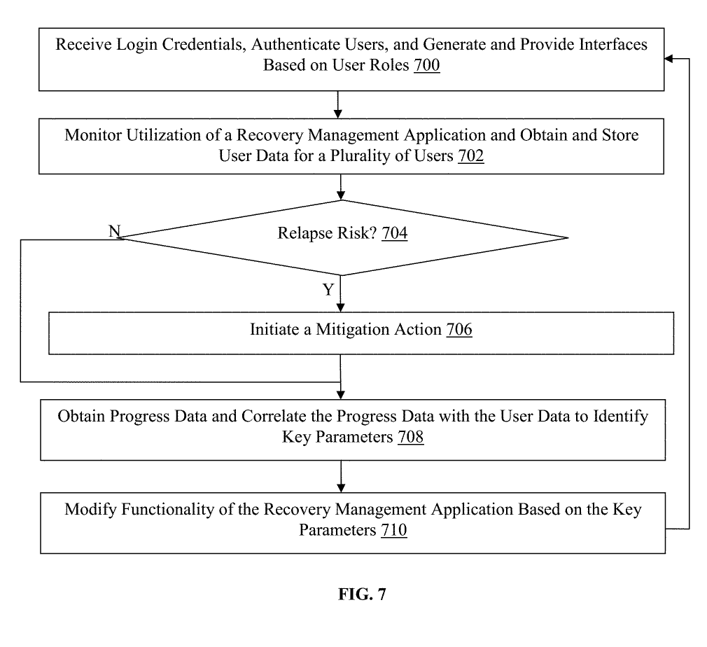

The learning module 28 is configured to monitor utilization of the recovery management application and process the content of the user information database in order to identify key parameters (e.g., a portion of the user data and one or more associated thresholds) that can be used to automatically modify functionality of, or otherwise reconfigure, the recovery management application so as to improve outcomes for users of the recover management application. Accordingly, the recovery management server device 12 delivers an experiential learning program via the recovery management application, and gathers and processes data relating to efficacy to more effectively facilitate the process of moving towards sobriety and recovery from addiction for users. The operation of the learning module 28 will be described and illustrated in more detail later with reference to FIG. 7.

The media content 30 in this example includes a plurality of videos that together comprise a multi-step or multi-part program that is directed toward providing inspiration and other useful information to users of the recovery management application 12. The media content can be retrieved via the recovery management application, or via another portal or interface provided by the recovery management server device 12, for example. In one example, the recovery management application can automatically process part of the user data for a particular user that is retrieved from the user information database 26 (e.g., historical mood data) in order to identify, retrieve, and output particular portions of the media content 30 that may be most effective for supporting a user of the recovery management application at the particular time of the user's current engagement with the recovery management application, as described and illustrated in more detail later.

The communication interface 22 in the recovery management server device 12 is used to operatively couple and communicate between the recovery management server device 12 and the client devices 14(1)-14(n) via the communication network(s) 16, although other types and numbers of connections and configurations can also be used. By way of example only, the communication networks 16 can include one or more local area networks or wide area networks, for example, and can use TCP/IP over Ethernet and industry-standard protocols, including hypertext transfer protocol (HTTP) and secure HTTP (HTTPS), although other types and numbers of communication networks, such as a direct connection, modems and phone lines, e-mail, and wireless and hardwire communication technology, each having their own communications protocols, can also be used.

The client devices 14(1)-14(n) in this example each include a processor 32(1)-32(n), a memory 34(1)-34(n), and a communication interface 36(1)-36(n), and optionally include an input device and/or a display device, which are coupled together by a bus or other communication link. The client devices 14(1)-14(n) can also have other numbers and types of systems, devices, components, and elements in other configurations and locations. The client devices 14(1)-14(n) can be mobile computing devices, smartphones, tablets, laptops, desktop computers, or any combination thereof, for example.

The processor(s) 32(1)-32(n) in the client devices 14(1)-14(n) execute a program of stored instructions for one or more aspects of the present technology, as described and illustrated by way of the examples herein. Additionally, other types and numbers of processing devices and configurable hardware logic could be used and the processor(s) 32(1)-32(n) could execute other numbers and types of programmed instructions.

The memory 34(1)-34(n) in the client devices 14(1)-14(n) stores these programmed instructions for one or more aspects of the present technology as described and illustrated herein, although some or all of the programmed instructions could be stored and executed elsewhere. A variety of different types of memory storage devices, such as a RAM, ROM, flash, SSDs, HDDs, or other computer readable medium which is read from and written to by a magnetic, optical, or other reading and writing system that is coupled to the processor(s), can be used for the memory 34(1)-34(n).

In this example, the memory 34(1)-34(n) includes a recovery management application 38(1)-38(n), which optionally can be retrieved from the recovery management server device 12 and stored locally. The recovery management applications 38(1)-38(n) can interface with the recovery management server device 12 via the communication network(s) 16 in this example. In other examples, the recovery management server device 12 can host a web-based portal by which users of the client devices 14(1)-14(n) can access the recovery management applications 38(1)-38(n).

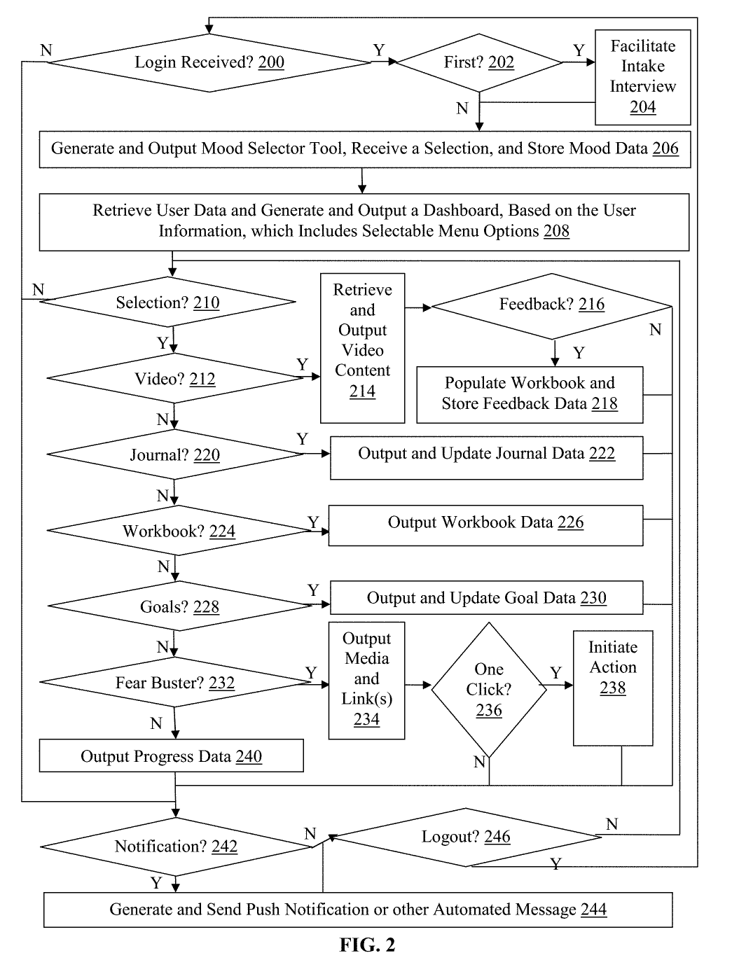

The recovery management applications 38(1)-38(n) generally provide a portal for users of the client devices 14(1)-14(n) to receive and store information that is utilized to improve the likelihood that the users will maintain a path of sobriety. Accordingly, each of the recovery management applications 38(1)-38(n) is an interface to bring forth and accentuate several factors in the path between addiction and sobriety. The operation of each of the recovery management applications 38(1)-38(n) is described and illustrated in more detail later with reference to FIG. 2.

Although examples of the recovery management server device 12 and client devices 14(1)-14(n), which are coupled together via the communication network(s) 16 are described herein, other types and/or numbers of computer systems or computing devices can also be used. It is to be understood that the devices and systems of the examples described herein are for exemplary purposes, as many variations of the specific hardware and software used to implement the examples are possible, as will be appreciated by those skilled in the relevant art(s).

In addition, two or more computing systems or devices can be substituted for any one of the systems in any embodiment of the examples. The examples may also be implemented on computer device(s) that extend across any suitable network using any suitable interface mechanisms and communications technologies, including by way of example only telecommunications in any suitable form (e.g., voice and modem), wireless communications media, wireless communications networks, cellular communications networks, G3 communications networks, Public Switched Telephone Network (PSTNs), Packet Data Networks (PDNs), the Internet, intranets, or combinations thereof.

The examples may also be embodied as one or more non-transitory computer readable media having programmed instructions stored thereon for one or more aspects of the present technology, as described and illustrated by way of the examples herein. The programmed instructions, when executed by one or more processors, cause the processors to carry out the steps necessary to implement one or more methods of the examples, as described and illustrated herein.

An exemplary method for facilitating addiction recovery services will now be described with reference to FIGS. 1-9. Referring more specifically to FIG. 2, a flowchart of an exemplary method for integrating relevant multimedia content and providing notifications and other resources to assist users in maintaining sobriety will now be described. In step 200 in this example, one of the client devices 14(1)-14(n) executing one of the recovery management applications 38(1)-38(n) determines whether authenticated login credentials are received from a user. If the one of the client devices 14(1)-14(n) determines that authenticated login credentials are received from a user, then the Yes branch is taken to step 202.

In step 202, the one of the client devices 14(1)-14(n) executing the one of the recovery management applications 38(1)-38(n) determines whether the login is a first login for the user associated with the authenticated credentials. If the one of the client devices 14(1)-14(n) determines that the login is the first login for the user, then the Yes branch is taken to step 204.

In step 204, the one of the client devices 14(1)-14(n) executing the one of the recovery management applications 38(1)-38(n) facilitates an intake interview and obtains intake interview data based on responses input via the intake interview. A score can be generated based on the intake interview data and, accordingly, the intake interview operates to establish a baseline for individual users of the one of the recovery management applications 38(1)-38(n). The intake interview data and/or the score can sent to the recovery management server device 12 to be stored as part of the user data for the user in the user information database 26, for example.

While the intake interview is initiated as part of a first login in this example, the intake interview can be provided separate from a login attempt and other methods of obtaining the intake interview data can also be used in other examples. Subsequent to facilitating the intake interview in step 204, or if the one of the client devices 14(1)-14(n) determines in step 202 that the login is not the first login for the user and the No branch is taken, then the client device proceeds to step 206.

In step 206, the one of the client devices 14(1)-14(n) executing the one of the recovery management applications 38(1)-38(n) generates and output a mood selector tool, receives a selection of a particular mood, and stores mood data reflecting the particular selection. The mood data can be sent to the recovery management server device 12 and stored in the user information database 26 as associated with the user. Accordingly, the user information database 26 can store historical mood data for each user of one of the recovery management applications 38(1)-38(n).

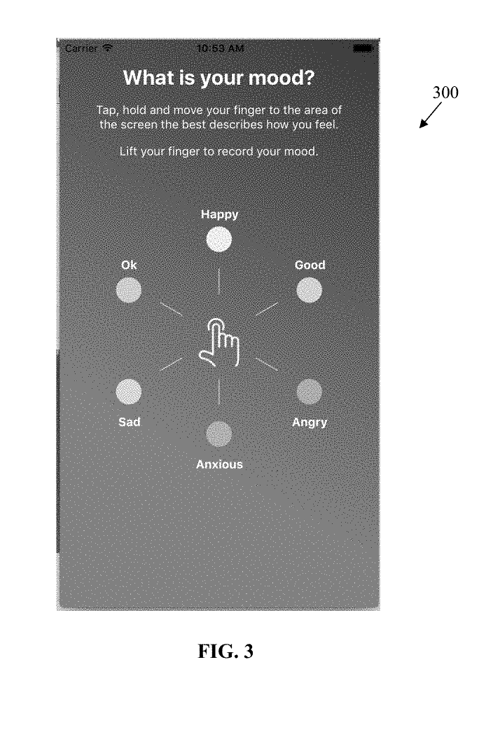

Referring more specifically to FIG. 3, a screenshot of an exemplary mood selector tool 300 is illustrated. In this example, the mood selector tool 300 facilitates selection of one of six displayed moods, although any number of moods can be selected in other examples. The tracking of mood over time can facilitate providing more relevant multimedia content to users in order to more effectively assist the users with respect to sobriety, as well as for other purposes in other examples.

Referring back to FIG. 2, in step 208, the one of the client devices 14(1)-14(n) executing the one of the recovery management applications 38(1)-38(n) retrieves user data for the particular user of the one of the client devices 14(1)-14(n) and generates and outputs a dashboard, based on the user information, which includes selectable menu options associated with particular modules. For example, the dashboard can include links to a thought of the day, which can be in an interactive and/or video format, a bird's eye view of goals, and/or recent journal entries.

In step 210, the one of the client devices 14(1)-14(n) executing the one of the recovery management applications 38(1)-38(n) determines whether a selection of one of the menu options has been received. While the menu options on the dashboard are selected to initiate a module in this example, one or more of the modules can be initiated in other ways (e.g., via a quick menu) and the dashboard can include other types of information and selections. If the one of the client devices 14(1)-14(n) determines that a selection has been received, then the Yes branch is taken to step 212.

In step 212, the one of the client devices 14(1)-14(n) executing the one of the recovery management applications 38(1)-38(n) determines whether the selection was of a link to a video module. The one of the recovery management applications 38(1)-38(n) can be configured to retrieve videos from the media content of the recovery management server device 12, and display the videos via the video module in this example. The videos can be a recommended amount of inspirational video content for a particular day or time of day, for example. If the one of the client devices 14(1)-14(n) determines that the selection was of a link to a video module, then the Yes branch is taken to step 214.

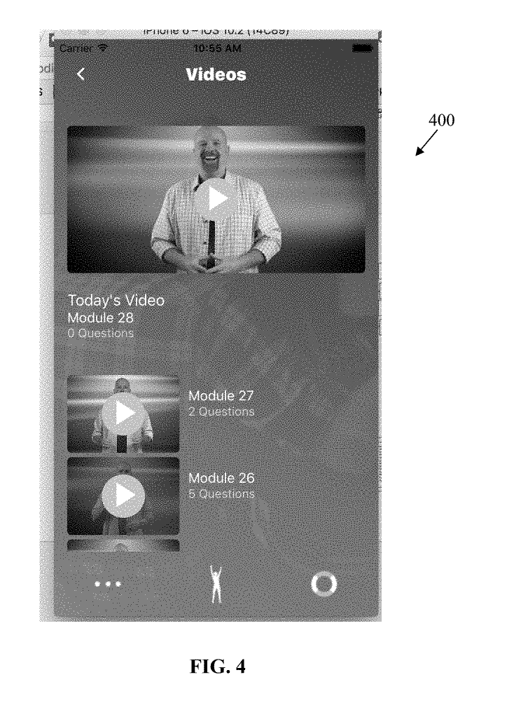

In step 214, the one of the client devices 14(1)-14(n) executing the one of the recovery management applications 38(1)-38(n) retrieves and outputs video content, such as from the media content 30 stored by the recovery management server device 12, for example. Referring more specifically to FIG. 4, a screenshot of an exemplary video module 400 is illustrated. In this example, the videos are interactive videos that initiate questions and facilitate the input of answers. Optionally, the video content that is initially offered to a user via the video module can be determined based on an analysis of the user data stored in the user information database 26 for the user. For example, if a user is consistently depressed based on the historical mood data, then videos categorized as being gratitude videos can be retrieved and output.

Referring back to FIG. 2, in step 216, the one of the client devices 14(1)-14(n) executing the one of the recovery management applications 38(1)-38(n) determines whether feedback has been received in response to one or more questions posed as part of an output interactive video. If the one of the client devices 14(1)-14(n) determines that feedback has been received, then the Yes branch is taken to step 218. In step 218, the one of the client devices 14(1)-14(n) executing the one of the recovery management applications 38(1)-38(n) populates a workbook for a user and stores feedback data as associated with the user in the user information database 26. Subsequent to populating the workbook, or if the one of the client devices 14(1)-14(n) determines that feedback has not been provided and the No branch is taken from step 216, then the one of the client devices 14(1)-14(n) proceeds to step 242 in this example, which is described and illustrated in more detail later, although there may be several iterations of one or more of steps 214-218 in other examples.

Referring back to step 212, if the one of the client devices 14(1)-14(n) determines that the selection was not of a link to the video module, then the No branch is taken to step 220. In step 220, the one of the client devices 14(1)-14(n) executing the one of the recovery management applications 38(1)-38(n) determines whether the selection was of a link to a journal module. If the one of the client devices 14(1)-14(n) determines that the selection was of a link to the journal module, then the Yes branch is taken to step 222.

In step 222, the one of the client devices 14(1)-14(n) executing the one of the recovery management applications 38(1)-38(n) outputs and updates journal data that was included in the user data obtained in step 208. Optionally, a user can add images to journal entries and set images as background images for the one of the recovery management applications 38(1)-38(n). Accordingly, the journal module is configured to retrieve and output journal data included in the user data, receive journal entries, and update the journal data based on the journal entries. Subsequent to facilitating interaction with the journal module, the one of the client devices 14(1)-14(n) optionally proceeds to step 242.

However, if the one of the client devices 14(1)-14(n) determines in step 220 that the selection was not of a link to the journal module, then the No branch is taken to step 224. In step 224, the one of the client devices 14(1)-14(n) executing the one of the recovery management applications 38(1)-38(n) determines whether the selection was of a link to a workbook module. The workbook module in this example is configured to retrieve and output the feedback data submitted via the interactive questions of the video module 400. Accordingly, if the one of the client devices 14(1)-14(n) determines that the selection was of a link to the workbook module, then the Yes branch is taken to step 226.

In step 226, the one of the client devices 14(1)-14(n) executing the one of the recovery management applications 38(1)-38(n) outputs workbook data. The workbook data can include historical responses to interactive videos as stored in the user information database 26, for example. Subsequent to facilitating interaction with the workbook module, the one of the client devices 14(1)-14(n) optionally proceeds to step 242. However, if the one of the client devices 14(1)-14(n) determines in step 224 that the selection was not of a link to the workbook module, then the No branch is taken to step 228.

In step 228, the one of the client devices 14(1)-14(n) executing the one of the recovery management applications 38(1)-38(n) determines whether the selection was of a link to a goals module. The goals module is configured to retrieve and output goal data included in the user data stored in the user information database 26, receive one or more new goals, and update the goal data based on the new goals. Goals can optionally be generated or retrieved automatically by the one of the recovery management applications 38(1)-38(n) based on analysis of the user data for a particular user. If the one of the client devices 14(1)-14(n) determines that the selection was of a link to the goals module, then the Yes branch is taken to step 230.

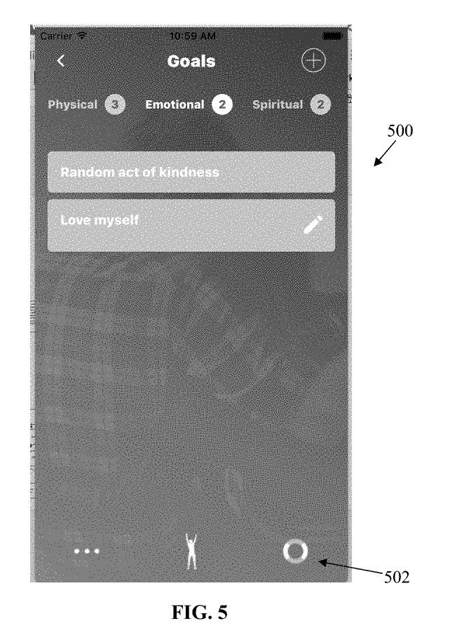

In step 230, the one of the client devices 14(1)-14(n) executing the one of the recovery management applications 38(1)-38(n) outputs and updates goal data. Referring more specifically to FIG. 5, a screenshot of an exemplary goals module 500 is illustrated. In this example, the goals module 500 is configured to display physical, emotional, and spiritual goals, although other types of goals can be used in other examples. Physical goals can include exercise, meals, learning skills, or being productive. Spiritual goals can relate to praying, meditating, attending services, or breathing exercises. Additionally, emotional goals can include random acts of kindness, communicating with family and/or friends, striving for a new achievement, or attending a group therapy meeting, for example. The goals module can facilitate display, editing of, and completing of goals, for example. Subsequent to facilitating interaction with the goals module 500, the one of the client devices 14(1)-14(n) optionally proceeds to step 242.

However, if the one of the client devices 14(1)-14(n) determines in step 228 that the selection was not of a link to the goals module 500, then the No branch is taken to step 232. In step 232, the one of the client devices 14(1)-14(n) executing the one of the recovery management applications 38(1)-38(n) determines whether the selection was of a link to a fear buster module. The fear buster module in this example is configured to retrieve and output inspirational media content and display a selectable one-click connection configured to, when selected, initiate a communication to a preestablished third party contact. If the one of the client devices 14(1)-14(n) determines that the selection was of a link to the fear buster module, then the Yes branch is taken to step 234.

In step 234, the one of the client devices 14(1)-14(n) executing the one of the recovery management applications 38(1)-38(n) outputs media and/or link(s) that are targeted to immediately assists the user of the one of the client devices 14(1)-14(n) in dealing with the current fear so as to stay on the path to sobriety. In one example, the fear buster module can include a one-click link that automatically initiates a phone call or other type of communication to an established third party contact. In other examples, such as illustrated in FIG. 5, a one-click link 502 can be provided as part of a quick menu that is accessible irrespective of the current module that is displayed on the one of the client devices 14(1)-14(n), and the one-click link 502 can be provided in other manners in other examples.

Accordingly, in step 236 in this particular example, the one of the client devices 14(1)-14(n) executing the one of the recovery management applications 38(1)-38(n) determines whether the one-click functionality is initiated by the user and, if it is, proceeds to step 238. In step 238, the one of the client devices 14(1)-14(n) executing the one of the recovery management applications 38(1)-38(n) automatically engages other functionality provided by an operating system of the one of the client devices 14(1)-14(n) (e.g., mobile calling functionality) to initiate a communication to a third party based on established contact information for the third party stored in a profile for the user as part of the user data at the recovery management server device 12. The third party can be a key contact, therapist, doctor, or counselor, for example.

In yet other examples, selection of the one-click link 502 can result in the one of the recovery management applications 38(1)-38(n) proceeding directly to a journal entry or functionality provided by another module. Subsequent to initiating the communication in this example, or if the one of the client devices 14(1)-14(n) determines that the one-clock functionality has not been selected and the No branch is taken from step 236, then the one of the client devices 14(1)-14(n) optionally proceeds to step 242.

Referring back to step 232, if the one of the client devices 14(1)-14(n) determines that the selection was not of a link to a fear buster module, then the No branch is taken to step 240. In step 240, the one of the client devices 14(1)-14(n) executing the one of the recovery management applications 38(1)-38(n) outputs a progress module. The progress module in this example is configured to retrieve and graphically output historical mood data included in the user data, as well as one or more statistical indicators based on other portions of the user data, although other information can also be provided by the progress module 600 in other examples.

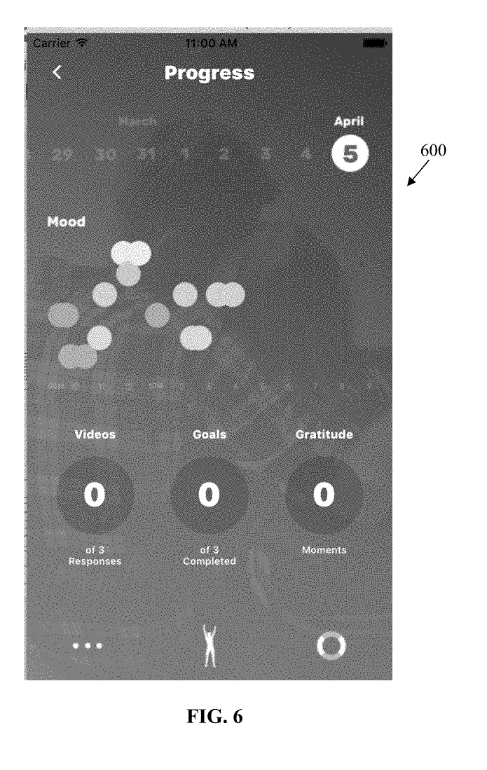

Accordingly, the progress module illustrates progress over time of user mood rankings, provides bird's eye view of progress in video watching for the day, goals for the day, and gratitude journal entries, for example. The one of the client devices 14(1)-14(n) can analyze the user data retrieved in step 208 in order to generate and output the progress data. Referring more specifically to FIG. 6, a screenshot of an exemplary progress module 600 is illustrated. In this example, historical mood data is graphed to show changes over time. Additionally, statistical indicators relating to videos, goals, and gratitude are provided, although other information can be generated and/or displayed by the progress module 600 in other examples. Subsequent to facilitating interaction with the progress module, the one of the client devices 14(1)-14(n) optionally proceeds to step 242.

Accordingly, if the selection is not of a link associated with a video, journal, workbook, goals, or fear buster module, then the selection is of a link to a progress module 600 in this example, although in other examples other types and numbers of modules can be provided and selected. By way of example only, a profile module can facilitating mapping, finding, setting, and saving meeting locations, adding editing, and updating contacts, including contact(s) associated with the one-click link, and managing settings such as screen update info, passwords, notifications, reminders, and signing out, for example.

Subsequent to interacting with one of the selected modules, or if the one of the client devices 14(1)-14(n) determines in step 200 that login credentials were not received and the No branch is taken, or that a selection of a menu option or link was not received in step 210 and the No branch is taken, then the one of the client devices 14(1)-14(n) proceeds to step 242 in this example. In step 242, the one of the client devices 14(1)-14(n) executing the one of the recovery management applications 38(1)-38(n) determines whether any notifications are required. While illustrated serially in the flowchart illustrated in FIG. 2, in some examples, the one of the client devices 14(1)-14(n) can determine whether any notification is required in parallel with performing any of steps 206-240. Additionally, one or more other of steps 200-246 can be performed in parallel and/or in other orders in other examples. Accordingly, if the one of the client devices 14(1)-14(n) determines that a notification is required, then the Yes branch is taken to step 244.

In step 244, the one of the client devices 14(1)-14(n) executing the one of the recovery management applications 38(1)-38(n) generates and sends a push notification or another automated message. Accordingly, the notifications can be push notifications or messages communicated via SMS or e-mail to contact(s) associated with a user, for example. In one example, the client device executing the one of the recovery management applications 38(1)-38(n) can determine whether a goal, stored as part of the goals data associated with a user, has not been complete within an associated specified time period, requiring initiation of a push notification reminder to the user. If the goal is not completed within another time period, the one of the recovery management applications 38(1)-38(n) initiates an automated e-mail notification to a sponsor/counselor in this example. Other types and number of notifications and/or triggers can be used in other examples.

Subsequent to generating and sending the push notification or other automated message, or if the one of the client devices 14(1)-14(n) determines that a notification is not required in step 242 and the No branch is taken, then the one of the client devices 14(1)-14(n) proceeds to step 246. In step 246, the one of the client devices 14(1)-14(n) executing the one of the recovery management applications 38(1)-38(n) determines whether the user has logged out. If the one of the client devices 14(1)-14(n) determines that a logout has not been received, then the No branch is taken back to step 210. Accordingly, while a user is logged in, the one of the client devices 14(1)-14(n) effectively waits to determine whether a selection of a link provided via the dashboard has been received, a notification is required, or a logout has occurred.

However, if the one of the client devices 14(1)-14(n) determines in step 246 that a logout has been received, then the Yes branch is taken back to step 200. Accordingly, once logged out, the one of the client devices 14(1)-14(n) effectively waits for a login request to be received or a notification to be required. Other methods of launching and exiting the one of the recovery management applications 38(1)-38(n) can also be used in other examples.

Referring more specifically to FIG. 7, a flowchart of an exemplary method for automated updating of a recovery management application based on machine learning to improve efficacy and associated user outcomes is illustrated. In step 700, in this example, the recovery management server device 12 receives login credentials, authenticates users, and generates and provides interfaces based on user roles.

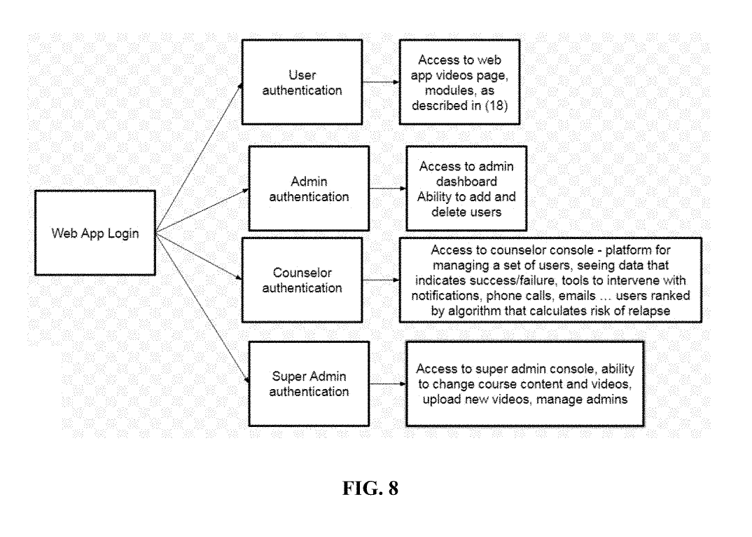

Referring to FIG. 8, a block diagram illustrating various exemplary portals or interfaces provided by the recovery management server device 12 is illustrated. In this particular example, a web application login request can include credentials associated with different roles including user, administrator, counselor, and super administrator roles, although other types of roles can be used in other examples. Following a user authentication, the recovery management server device 12 can provide access to a web version of the recovery management application 38 or directly to a application videos page with links to portions of the stored media content.

Following an administrator login, the recovery management server device 12 can provide access to an administrator dashboard, which allows an administrator to add and delete users, among other functions. Additionally, the recovery management server device 12 can provide access to an interface for a super administrator that allows the super administrator to change and upload media content (e.g., videos) as well as manage administrators, for example.

Counselors can access the recovery management server device 12 to manage a set of associated or linked users. Counselors can manage their associated users by retrieving portions of the user data associated with those users (e.g., successes or failures of the users), interacting with tools that facilitate interventions (e.g., notifications or messages), or retrieving a ranking of their associated users with respect to a risk of relapse, for example, although other types of information and/or tools can be provided to counselors in other examples.

Referring back to FIG. 7, in step 702, the recovery management server device 12 monitors utilization of the recovery management applications 38(1)-38(n) executed by the client devices 14(1)-14(n) and obtains and stores user data for a plurality of users. As described and illustrated earlier, the user data can include intake interview data, mood data, feedback/workbook data, journal entry data, goals data, progress data, statistical usage and participation data, and profile data, and other types of user data can also be obtained and stored in the user information database 26.

In step 704, the recovery management server device 12 optionally determines whether a relapse risk value associated with a user exceeds an established threshold value. In this example, the recovery management server device 12 generates relapse risk value for one or more of the users for which user data is stored in the user information database 26. The recovery management server 12 can determine (e.g., based on historical progress data and user outcomes), factors (e.g. portions of the user data) that correlate with an increased risk of relapse. For example, statistical data showing a relatively low check-in rate combined with consistent sad/depressed mood states in the mood data may indicate an increased risk of relapse. Accordingly, if the recovery management server device 12 determines that a relapse risk value for a user exceeds a threshold, then the Yes branch is taken to step 706.

In step 706, the recovery management server device 12 initiates a mitigation action with respect to the particular user. The mitigation action can include an automated message to a counselor or establishing a schedule or push notifications to encourage user engagement with the recovery management application or a particular module thereof, for example, although other types and number of mitigation actions can also be initiated. In another example, the recovery management server device 12 can generate a custom coaching/counseling console for a counselor to efficiently intervene in relatively high risk moments for a user, thereby optimizing the process of counseling and leveraging the user data to make counseling more effective, quicker, more efficient and more impactful.

Subsequent to initiating the mitigation action, or if the recovery management server device 12 determines in step 704 that the relapse risk value does not exceed the threshold for any user and the No branch is taken, then the recovery management server device 12 proceeds to step 708. In step 708, the recovery management server device 12 obtains progress data and correlates the progress data with the user data to identify key parameters (e.g., portions of the user data). The progress data can be based on a period of sobriety, a number of relapse events, or any other type of information indicative of progress or an outcome for a user.

In step 710, the recovery management server device 12 modifies the functionality of the recovery management applications 38(1)-38(n) for one or more users based on the key parameters. Accordingly, through use of the recovery management applications 38(1)-38(n), and associated video content program, the recovery management server device 12 effectively gathers relevant user data necessary to make predictive analysis of whether certain aspect (e.g., modules) work and what forms of interventions and encouragement make a difference for users. The gathering of the user data can inform the way(s) that one or more of the recovery management applications 38(1)-38(n) intervenes in key moments, when they intervene, and how they intervene, resulting in both guidance towards how the content and responsiveness will evolve, as well as a means for the approach of the recovery management applications 38(1)-38(n) to tailor itself to individual(s).

In one particular example, the recovery management server device 12 can identify certain ranges of scores for an intake interview as a key parameter correlating with outcomes, and can tailor functionality of one or more of the recovery management applications 38(1)-38(n) accordingly for particular subsets of users. Other types and numbers of key parameters can also be used in other examples.

Accordingly, with this technology, users recovering from addiction can be provided a more effective means for maintaining a path of sobriety. This technology provides media content and other guidance and modules, via a recovery management application, based on an analysis of particular parameters and factors for certain users. This technology is advantageously adaptive and capable of transforming the functionality of the recovery management application in order to facilitate a positive outcome for users.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method for facilitating addiction recovery services implemented by one or more client devices, the method comprising:
authenticating a user in response to a received login request, outputting a mood selector tool, and retrieving current mood data based on a received interaction with the mood selector tool;
obtaining user data for the user and generating and outputting a dashboard comprising a plurality of selectable menu options;
receiving a selection of one of the selectable menu options, outputting one of a plurality of modules corresponding to the one of the selectable menu options, the one of the modules configured to provide content based on at least a portion of the user data and the current mood data; and
determining when a notification condition exists and outputting a push notification or sending an automated message to a third party, when the determining indicates that a notification condition exists.

2. The method of claim 1, wherein the modules comprise one or more of:
a video module configured to retrieve and output recommended video content associated with particular portions of a predefined program, each having associated interactive questions, and receive and store feedback data submitted via the interactive questions;
a journal module configured to retrieve and output journal data included in the user data, receive journal entries, and update the journal data based on the journal entries;
a workbook module configured to retrieve and output the feedback data submitted via the interactive questions of the video module;
a goals module configured to retrieve and output goal data included in the user data, receive one or more new goals, and update the goal data based on the new goals;
a fear buster module configured to retrieve and output inspiration media content, display a selectable one-click connection configured to, when selected, initiate a communication to a preestablished third party contact; or
a progress module configured to retrieve and graphically output historical mood data included in the user data as well as one or more statistical indicators.

3. The method of claim 1, wherein the user data comprises information previously submitted via one or more of the modules, intake interview data, or statistical usage data.

4. A client device, comprising memory comprising programmed instructions stored thereon and one or more processors configured to be capable of executing the stored programmed instructions to:
authenticate a user in response to a received login request, output a mood selector tool, and retrieve current mood data based on a received interaction with the mood selector tool;
obtain user data for the user and generate and output a dashboard comprising a plurality of selectable menu options;
receive a selection of one of the selectable menu options, output one of a plurality of modules corresponding to the one of the selectable menu options, the one of the modules configured to provide content based on at least a portion of the user data and the current mood data; and
determine when a notification condition exists and output a push notification or send an automated message to a third party, when the determining indicates that a notification condition exists.

5. The client device of claim 4, wherein the modules comprise one or more of:
a video module configured to retrieve and output recommended video content associated with particular portions of a predefined program, each having associated interactive questions, and receive and store feedback data submitted via the interactive questions;
a journal module configured to retrieve and output journal data included in the user data, receive journal entries, and update the journal data based on the journal entries;
a workbook module configured to retrieve and output the feedback data submitted via the interactive questions of the video module;
a goals module configured to retrieve and output goal data included in the user data, receive one or more new goals, and update the goal data based on the new goals;
a fear buster module configured to retrieve and output inspiration media content, display a selectable one-click connection configured to, when selected, initiate a communication to a preestablished third party contact; or
a progress module configured to retrieve and graphically output historical mood data included in the user data as well as one or more statistical indicators.

6. The client device of claim 4, wherein the user data comprises information previously submitted via one or more of the modules, intake interview data, or statistical usage data.

7. A non-transitory computer readable medium having stored thereon instructions for facilitating addiction recovery services comprising executable code which when executed by one or more processors, causes the one or more processors to:
authenticate a user in response to a received login request, output a mood selector tool, and retrieve current mood data based on a received interaction with the mood selector tool;
obtain user data for the user and generate and output a dashboard comprising a plurality of selectable menu options;
receive a selection of one of the selectable menu options, output one of a plurality of modules corresponding to the one of the selectable menu options, the one of the modules configured to provide content based on at least a portion of the user data and the current mood data; and
determine when a notification condition exists and output a push notification or send an automated message to a third party, when the determining indicates that a notification condition exists.

8. The non-transitory computer readable medium of claim 7, wherein the modules comprise one or more of:
a video module configured to retrieve and output recommended video content associated with particular portions of a predefined program, each having associated interactive questions, and receive and store feedback data submitted via the interactive questions;
a journal module configured to retrieve and output journal data included in the user data, receive journal entries, and update the journal data based on the journal entries;
a workbook module configured to retrieve and output the feedback data submitted via the interactive questions of the video module;
a goals module configured to retrieve and output goal data included in the user data, receive one or more new goals, and update the goal data based on the new goals;
a fear buster module configured to retrieve and output inspiration media content, display a selectable one-click connection configured to, when selected, initiate a communication to a preestablished third party contact; or
a progress module configured to retrieve and graphically output historical mood data included in the user data as well as one or more statistical indicators.

9. The non-transitory computer readable medium of claim 7, wherein the user data comprises information previously submitted via one or more of the modules, intake interview data, or statistical usage data.
